SEQUENCE LISTING (11 sequences)

SEQ ID NO: 1 - protein, 9 aa, Artificial Sequence
  Chemically Synthesized
GRTGRRNAI

SEQ ID NO: 2 - protein, 11 aa, Articifical Sequence
  Chemically Synthesized
GRTGRRNAIHD

SEQ ID NO: 3 - protein, 20 aa, Artificial Sequence [sequence omitted]

SEQ ID NO: 4 - protein, 17 aa, Artificial Sequence
  Chemically Synthesized
TYADFIASGRTGRRNAI

SEQ ID NO: 5 - protein, 15 aa, Artificial Sequence
  Chemically Synthesized
GKGRGLSLSAFSWGA

SEQ ID NO: 6 - protein, 7 aa, Artificial Sequence
  Chemically Synthesized
RKRARKE

SEQ ID NO: 7 - protein, 7 aa, Artificial Sequence
  Chemically Synthesized
RKRSRAE

SEQ ID NO: 8 - protein, 7 aa, Artificial Sequence
  Chemically Synthesized
RKRSRKE

SEQ ID NO: 9 - protein, 13 aa, Artificial Sequence
  Chemically Synthesized
KKALRRQEAVDAL

SEQ ID NO: 10 - protein, 13 aa, Artificial Sequence
  Chemically Synthesized
KKALRRQEAVDAL

SEQ ID NO: 11 - protein, 5 aa, Artificial Sequence
  Chemically Synthesized
YEEIE

(19) United States
(12) Patent Application Publication
(10) Pub. No.: US 2009/0092566 A1
(43) Pub. Date: Apr. 9, 2009

(54) SELF-TANNING COSMETIC COMPOSITIONS AND METHODS

(76) Inventors: Peter J. Lentini, West Babylon, NY (US); Paolo Li Giacomoni, Commack, NY (US)

Correspondence Address: Julie Blackburn, Suite 345 South, 155 Pinelawn Road, Melville, NY, 11747, US

(21) Appl. No.: 11/869,297

(22) Filed: Oct. 9, 2007

(51) Int. Cl. A61K 8/35; A61Q 17/04

(52) U.S. Cl. 424/59

(57) ABSTRACT

A self tanning composition containing at least one self tanning agent and at least one kinase inhibitor operable to inhibit the kinase induced phosphorylation of the self tanning agent; a method for increasing the efficacy of self tanning agents, and a method for tanning skin.

### TECHNICAL FIELD

The invention is in the field of cosmetic compositions for application to skin to provide darkening of the skin color to give the appearance of a tan.

### BACKGROUND OF THE INVENTION

Most self-tanning products currently on the market contain dihydroxyacetone, or DHA. When DHA is applied to skin it causes a skin darkening that gives the appearance of a tan. The darkening effects of DHA were discovered in the 1950's by a physician who was treating children affected by glycogen storage disease with large doses of DHA. Some of the material accidentally came into contact with skin and resulted in brown spots. Further tests of DHA in aqueous solution demonstrated consistent skin darkening properties. DHA causes skin darkening by reacting with the free amino groups in skin proteins or amino acids in what is referred to as the Maillard Reaction—the reaction of amines with sugars to create brown colored compounds. A DHA tan is superficial and affects only the stratum comeum.

Kinases, also known as phosphotransferases, are enzymes that transfer phosphate groups from high energy donor molecules to substrate molecules in what is referred to as a phosphorylation reaction. Protein kinases are kinases that modify the activity of proteins in this fashion. Phosphates of various types are present in skin. Protein kinases, present in skin cells, transfer phosphate groups to DHA and thereby reduce its effectiveness in darkening the skin. Because DHA binds to the amino groups present in skin, if the DHA is bound to phosphate groups it is not free to bind to amino groups present in skin and will not as effectively darken skin. In other words, phosphorylated DHA exhibits reduced effectiveness in darkening skin due to the activity of protein kinases which cause the phosphorylation.

### SUMMARY OF THE INVENTION

The invention is directed to a self tanning cosmetic composition comprising at least one skin tanning agent and at least one kinase inhibitor operable to inhibit the kinase induced phosphorylation of the skin tanning agent when the composition is applied to skin.

The invention is also directed to a method for increasing the efficacy of DHA in self tanning compositions comprising formulating the composition with at least one kinase inhibitor operable to inhibit the kinase induced phosphorylation of DHA.

The invention is also directed to a self tanning composition comprising at least one skin tanning agent operable to darken skin due to the Maillard Reaction, and at least one kinase inhibitor derived from a botanical extract having greater than about 0.1% ursolic acid, preferably from about 0.1 to 80%, more preferably from about 0.1 to 50% ursolic acid content, which is operable to inhibit the kinase induced phosphorylation of DHA.

A method for darkening the color of skin to provide the appearance of a tan by applying to the skin a composition comprising at least one skin tanning agent reactive with phosphorus and at least one kinase inhibitor operable to inhibit the kinase induced phosphorylation of the self tanning agent when the composition is applied to the skin.

### DETAILED DESCRIPTION

### I. Definitions

A. The term “kinase inhibitor” means an ingredient that inhibits the kinase induced phosphorylation of skin tanning ingredients, as further described herein, that cause the darkening of skin due to the Maillard Reaction.

B. The term “liquid” means a composition that is pourable at room temperature (25° C.)

C. The term “nonvolatile” means that the ingredient has a vapor pressure of less than about 2 mm. of mercury at 20° C.

D. The term % means percent by weight of the total composition unless otherwise indicated.

E. The term “semi-solid” means a composition that deforms upon application of shear.

F. The term “solid” means a composition that is capable of being self-supporting at room temperature, which may be found in the molded or pressed forms such as sticks, cakes, pressed, and the like.

G. The term “volatile” means that the ingredient has a vapor pressure of at least about 2 mm. of mercury at 20° C.

### II. Skin Tanning Ingredients

In addition to DHA (dihydroxyacetone), suitable skin tanning agents are ingredients that are known to provide browning on the skin via the Maillard Reaction and include sugars such as sucrose, glucose, fructose, xylose, erythrulose, etc. They are reactive with phosphorus, that is they are capable of being phosphorylated when exposed to phosphorus-containing ingredients found in skin.

Preferred is where the skin tanning ingredient is DHA either alone or in combination with the other ingredients. Suitable ranges of skin tanning ingredient range from about 0.001 to 50%, preferably from about 0.05 to 40%, more preferably from about 1 to 35% by weight of the total composition.

### III. The Kinase Inhibitor

The composition of the invention comprises at least one kinase inhibitor present in an amount sufficient to inhibit the kinase induced phosphorylation of DHA, either in whole or in part. Suggested ranges of kinase inhibitor may range from about 0.0001 to 25%, preferably from about 0.005 to 20%, more preferably from about 0.01 to 15% by weight of the total composition. Suitable kinase inhibitors may be in the form of botanical extracts, peptides, chemical compounds, mixtures of ingredients, and the like. The kinase inhibitor may inhibit the kinase induced phosphorylation of DHA by competitive inhibition or non-competitive inhibition. Kinase inhibitors that act by competitive inhibition include those that compete with DHA for phosphorylation or are preferentially phosphorylated over DHA. Suitable kinase inhibitors include, but are not limited to, those further described herein.

A. Botanical Extracts

Some types of botanical extracts are suitable kinase inhibitors in accordance with the invention. In one preferred embodiment, suitable kinase inhibitors are botanical extracts that contain significant amounts of ursolic acid, for example, from about 5 to 95%, preferably from about 10 to 85%, more preferably from about 10 to 75% by weight of the total extract.

Suitable botanical extracts may be derived from plants such as vegetables, fruits, flowers, trees, and the like, and include, but are not limited to:
(1) An extract obtained from the botanical Nonomuraea longicatena, also referred to as K252a
(2) An extract from Cladosporium cladosporioides, also referred to as Calphostin C
(3) Extract from Hypericum perforatum, also known as Hypericin
(4) Ligustrum Lucidum extract
(5) Eriobotrya Japonica extract
(6) Loquat Leaf Extract
(7) Betula Alba extract
(8) Fucus Vesiculosis (Bladderwrack) Extract
(9) Laminaria Digitata (Atlantic Kelp) Extract
(10) Origanum Majorana Extract
(11) Polylepsis Racemosa Extract
(12) Oldenlandia Diffusa Extract
(13) Leocerus Cardiaca (Motherwart) Extract
(14) Ocimum Tenuiflorum (Sacred Basil) Extract
(15) Pygeum Africanum Extract
(16) Frangula Bark Extract (6-Methyl-1,3,8-tri [symbol] hydroxy [symbol] anthra [symbol] quinine); and
(17) mixtures thereof.

B. Peptides

Also suitable as kinase inhibitors are various peptides or peptide derivatives, including but not limited to:
(1) Protein Kinase A inhibitor fragment 14-22, myristoylated trifluoroacetate salt, having the amino acid sequence: Myr-Gly-Arg-Thr-Gly-Arg-Arg-Asn-Ala-Ile-NH₂
(2) Protein kinase A inhibitor fragment 14-24 trifluoroacetate salt having the amino acid sequence: Gly-Arg-Thr-Gly-Arg-Arg-Asn-Ala-Ile-His-Asp-NH₂
(3) Protein Kinase A Inhibitor Fragment 5-24 amide acid salt having the amino acid sequence: Thr-Thr-Tyr-Ala-Asp-Phe-Ile-Ala-Ser-Gly-Arg-Thr-Gly-Arg-Arg-Asn-Ala-Ile-His-Asp-NH₂
(4) Protein Kinase Inhibitor 6-22 Amide, having the amino acid sequence Thr-Tyr-Ala-Asp-Phe-Ile-Ala-Ser-Gly-Arg-Thr-Gly-Arg-Arg-Asn-Ala-Ile-NH₂
(5) [Ala113]-MBP fragment 104-118, having the amino acid sequence: Gly-Lys-Gly-Arg-Gly-Leu-Ser-Leu-Ser-Ala-Phe-Ser-Trp-Gly-Ala
(6) Arg-Lys-Arg-Ala-Arg-Lys-Glu
(7) Arg-Lys-Arg-Ser-Arg-Ala-Glu
(8) Arg-Lys-Arg-Ser-Arg-Lys-Glu
(9) [Ala9]-Autocamtide 2 AIP, a peptide fragment having this amino acid sequence: Lys-Lys-Ala-Leu-Arg-Arg-Gln-Glu-Ala-Val-Asp-Ala-Leu
(10) Myr-Lys-Lys-Ala-Leu-Arg-Arg-Gln-Glu-Ala-Val-Asp-Ala-Leu-OH
(11) N-Acetyl-O-phosphono-Tyr-Glu-Glu-Ile-Glu,
(12) Protein Kinase C Inhibitor, Myristoylated; or
(13) mixtures thereof.

C. Compounds or Molecules

Also suitable as kinase inhibitors are a variety of chemical compounds or molecules, including but not limited to those set forth herein:
(1) Akt1/2, also referred to as Akt Inhibitor VIII trifluoroacetate salt hydrate, having the chemical name 1,3-Dihydro-1-(1-((4-(6-phenyl-1H-imidazo[4,5-g]quinoxalin-7-yl)phenyl)methyl)-4-piperidinyl)-2H-benzimidazol-2-one trifluoroacetate salt hydrate
(2) Aurora Kinase Inhibitor III, also referred to as cyclopropanecarboxylic acid {3-[4-(3-trifluoromethyl-phenylamino)-pyrimidin-2-ylamino]-phenyl}-amide
(3) Diacylglycerol Kinase Inhibitor I, having the chemical name 6-[2-[4-[(4-Fluorophenyl)phenylmethylene]-1-piperidin [symbol] yl]ethyl]-7-methyl-5H-thiazolo-[3,2-a]-pyrimidin-5-one
(4) Diacylglycerol Kinase Inhibitor II, having the chemical name 3-[2-[4-(bis(4-Fluorophenyl)methylene)-1-piperidinyl]ethyl]-2,3-dihydro-2-thioxo-4(1H)-quinazolinone
(5) Transforming Growth Factor-β Type I Receptor Kinase Inhibitor having the chemical name 4-[3-(2-pyridinyl)-1H-pyrazol-4-yl]-quinoline
(6) Aurora Kinase Inhibitor II, having the chemical name 4-(4′-Benzamidoanilino)-6,7-dimethoxyquinazoline hydrochloride
(7) Sphingosine Kinase Inhibitor V, having the chemical name 2-(3,4-Dihydroxy-benzylidene)-benzofuran-3-one
(8) DMAT, having the chemical name 2-Dimethylamino-4,5,6,7-tetrabromo-1H-benzimidazole
(9) Deguilin, having the chemical name (7aS,13aS)-13,13a-Dihydro-9,10-dimethoxy-3,3-dimethyl-3H-bis[1]benzopyrano[3,4-b:6′,5′-e]pyran-7(7aH)-one
(10) Erbstatin analog, having the chemical name methyl 2,5-dihydroxycinnamate
(11) 4-hydroxytamoxifen, having the chemical name: Z)-4-(1-[4-(Dimethylaminoethoxy)phenyl]-2-phenyl-1-butenyl)phenol trans-4-Hydroxytamoxifen
(12) 5,6-Dichloro-1-β-D-ribofuranosylbenzimidazole
(13) AMPK Inhibitor, having the chemical name: 6-[4-(2-Piperidin-1-ylethoxy)phenyl]-3-pyridin-4-ylpyrazolo[1,5-a]pyrimidine
(14) 7,8-Dihydroxycoumarin
(15) 7-Cyclopentyl-5-(4-phenoxyphenyl)-7H-pyrrolo[2,3d]pyrimidin-4-ylamine
(16) Rp-8-[(4-Chlorophenyl)thio]-cGMPS triethylammonium salt
(17) Rp-8-CPT-cGMPS, having the chemical description Rp-8-[(4-Chlorophenyl)thio]-guanosine-cyclic 3′,5′-hydrogen phosphorothioate triethylammonium salt
(18) N7-[(1′R,2′S,3′R,4′S)-2′,3′-dihydroxy-4′-aminocyclopentyl]-4-iodopyrrolopyrimidine dihydrochloride hydrate
(19) 3-(6-Methylpyridin-2-yl)-1-phenylthiocarbamoyl-4-quinolin-4-ylpyrazole
(20) 4-Amino-5-(3-bromophenyl)-7-(6-morpholinopyridin-3-yl)pyrido[2,3-d]pyrimidine
(21) N-Amidino-3,5-diamino-6-chloropyrazinecarboxamide hydrochloride
(22) 4′,5,7-Trihydroxyflavone
(23) N-(4-Methoxybenzyl)-N′-(5-nitro-1,3-thiazol-2-yl)urea
(24) 2-[[7-(3,4-Dimethoxyphenyl)imidazo[1,2-c]pyrimidin-5-yl]amino]pyridine-3-carboxamide hydrochloride
(25) 4-(2-Chlorophenyl)-1-ethyl-2-methyl-5-oxo-1,4,5,7-tetrahydrofuro[3,4-b]pyridine-3-carboxylic acid isopropyl ester
(26) 4-(2-Chlorophenyl)-1-ethyl-1,4-dihydro-6-methyl-2,3,5-pyridinetricarboxylic acid 5-isopropyl ester disodium salt hydrate
(27) (2′Z,3′E)-6-Bromoindirubin-3′-oxime
(28) Bisindolylmaleimide II, IV, V , VI, or VIII
(29) Bisindolylmaleimide VIII acetate salt
(30) Bisindolylmaleimide X hydrochloride
(31) Bisindolylmaleimide XI hydrochloride
(32) 1-(2,4-Dihydroxyphenyl)-3-(3,4-dihydroxyphenyl)-2-propen-1-one
(33) 2,2-Diphenyl-n-(2,2,2-trichloro-1-[3-(4-fluoro-3-nitrophenyl)thioureido]ethyl)acetamide
(34) 1,2-Dimethoxy-N-methyl(1,3)benzodioxolo(5,6-c)phenanthridinium chloride
(35) 2-(4-(4-chlorophenoxy)phenyl)-1H-benzimidazole-5-carboxamide
(36) N-(2-Aminoethyl)-5-chloroisoquinoline-8-sulphonamide dihydrochloride
(37) 4′,7-Dihydroxy [symbol] iso [symbol] flavone 7-Hydroxy-3-(4-hydroxy [symbol] phenyl)-4H-1-benzo [symbol] pyran-4-one 7-Hydroxy-3-(4-hydroxy [symbol] phenyl) [symbol] chromone
(38) 1,1′-Decamethylenebis(4-aminoquinaldinium) dichloride hydrate
(39) 4,4′,5,5′,6,6′-Hexahydroxydiphenic acid 2,6,2′,6′-dilactone
(40) 1,3,8-Tri [symbol] hydroxy-6-methyl [symbol] anthra [symbol] quinine
(41) 3-[1-[3-(Dimethylamino)propyl]-5-methoxy-1H-indol-3-yl]-4-(1H-indol-3-yl)-1H-pyrrole-2,5-dione
(42) Geldanamycin from Streptomyces hygroscopicus
(43) genestein
(44) 3-(N-[Dimethylamino]propyl-3-indolyl)-4-(3-indolyl)maleimide 3-[1-[3-(Dimethylamino)propyl]1H-indol-3-yl]-4-(1Hindol-3-yl)1H-pyrrole-2,5dione
(45) Bisindolylmaleimide I hydrochloride, or 3-[1-(Dimethylaminopropyl)indol-3-yl]-4-(indol-3-yl)maleimide hydrochloride
(46) N4-(1-Benzyl-1H-indazol-5-yl)-N6,N6-dimethyl-pyrido [symbol] [3,4-d] [symbol] pyrimidine-4,6-diamine
(47) 3-(3,5-Dibromo-4-hydroxybenzyliden)-5-iodo-1,3-dihydroindol-2-one
(48) 1-(5-Isoquinolinesulfonyl)-2-methylpiperazine Isoquinoline-5-sulfonic 2-methyl-1-piperazide
(49) 1-(5-Isoquinolinylsulfonyl)-2-methylpiperazine dihydro chloride
(50) N-[2-(Methylamino)ethyl]-5-isoquinolinesulfonamide hydrochloride
(51) N-[2-(p-Bromocinnamylamino)ethyl]-5-isoquinolinesulfonamide dihydrochloride
(52) 1-(5-Isoquinolinesulfonyl)piperazine hydrochloride
(53) N-(2-Guanidinoethyl)-5-isoquinolinesulfonamide hydrochloride
(54) 1-(5-Isoquinolinylsulfonyl)homopiperazine dihydrochloride
(55) Herbimycin A from Streptomyces hygroscopicus
(56) Hispidin
(57) α-Cyano-(3-methoxy-4-hydroxy-5-iodocinnamoyl)-(3′,4′-dihydroxyphenyl)ketone
(58) Indirubin-3′-monoxime
(59) (S)-5-Isoquinolinesulfonic acid 4-[2-[(5-isoquinolinylsulfonyl)methylamino]-3-oxo-3-(4-phenyl-1-piperazinyl)propyl]phenyl ester 1-[N,O-bis(5-Isoquinolinesulfonyl)-N-methyl-L-tyrosyl]-4-phenylpiperazine
(60) 2-[N-(4′-Methoxybenzenesulfonyl)]amino-N-(4′-chlorophenyl)-2-propenyl-N-methylbenzylamine phosphate
(61) N-[2-[[[3-(4′-Chlorophenyl)-2-propenyl]methylamino]methyl]phenyl]-N-(2-hydroxyethyl)-4′-methoxybenzenesulfonamide phosphate salt N-[2-[N-(4-Chlorocinnamyl)-N-methylaminomethyl]phenyl]-N-(2-hydroxyethyl)-4-methoxybenzenesulfonamide phosphate salt
(62) 5-(5-Ethyl-2-hydroxy-4-methoxyphenyl)-4-(4-methoxyphenyl)isoxazole
(63) Ellagic acid, or 4,4′,5,5′6,6′ hexahydroxydiphenic acid 2,6,2′6′ dilactone
(64) Olomoucine, or (2-(2-hydroxyethylamino)-6-benzylamino-0-methylpurine
(65) Adenosine 3′,5′-cyclic monophosphorothioate, 8 chloro, Rp isomer
(66) N-(2-aminoethyl)-5-chloronaphthalene-1-sulfonamide hydrochloride
(67) (5-isoquinolinesulfonyl)piperazine, 2 HCl
(68) Piceatannol, or trans 3-3′,4,5′-tetrahydroxystilbene
(69) Staurosporin
(70) Dihydroxyacetone phosphate
(71) Glyceraldehyde
(72) Glyceryl-3 phosphate: and
(73) mixtures thereof.

D. Vitamins or Vitamin Derivatives

Certain vitamins or vitamin derivatives are also suitable kinase inhibitors, such as Vitamin E acid succinate, and the like.

E. Sphingosine Derivatives

Suitable sphingosine derivatives are also good kinase inhibitors, including the following:
(1) 4-Sphingenine, also known as trans-D-erythro-2-Amino-4-octadecene-1,3-diol
(2) Saphingol, having the chemical name L-threo-Dihydrosphingosine

### IV. Other Ingredients

The self-tanner compositions of the invention may contain a variety of other ingredients. The compositions may be found in the anhydrous, emulsion, gel, solution, or suspension form. If present in the emulsion from, water-in-oil or oil-in-water emulsions may be suitable, and may include from about 0.01-99%, preferably from about 0.5-95%, more preferably from about 1-90% by weight of the total composition of water and from about 0.01-98%, preferably from about 0.1-95%, more preferably from about 0.5-90% by weight of the total composition of oil. The composition may contain a variety of other ingredients including but not limited to those set forth herein. If present in the solution or gel form, the composition may comprise from about 0.01-99% water and other optional ingredients. If present in the anhydrous form, the composition may contain waxes, oils, or humectants in the amounts set forth herein.

A. Volatile Oils
1. Volatile Silicones

Suitable volatile oils that may be used in the compositions of the invention generally have a viscosity ranging from about 0.5 to 5 centistokes 25° C. and include linear silicones, cyclic silicones, branched silicones, paraffinic hydrocarbons, or mixtures thereof. Cyclic silicones are of the general formula:

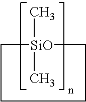

where n=3-6.

Linear volatile silicones in accordance with the invention have the general formula:

(CH₃)₃Si—O—[Si(CH₃)₂—O]ₙ—Si(CH₃)₃

where n=0, 1, 2, 3, 4, or 5, preferably 0, 1, 2, 3, or 4.

Branched volatile silicones are generally of the formula:

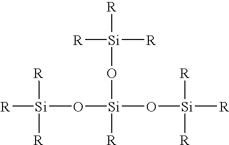

wherein R is C₁₋₄ alkyl, preferably methyl.

Linear and cyclic volatile silicones are available from various commercial sources including Dow Corning Corporation and General Electric. The Dow Coming volatile silicones are sold under the tradenames Dow Coming 244, 245, 344, and 200 fluids. These fluids comprise octamethylcyclotetrasiloxane, decamethylcyclopentasiloxane, dodecamethylcyclohexasiloxane and the like. Also suitable are linear volatile silicones such as hexamethyldisiloxane (viscosity 0.65 centistokes (abbreviated cst)), octamethyltrisiloxane (1.0 cst), decamethyltetrasiloxane (1.5 cst), dodecamethylpentasiloxane (2 cst) and mixtures thereof.

Suitable branched volatile silicones include methyl trimethicone, ethyl trimethicone, propyl trimethicone, butyl trimethicone and the like. Methyl trimethicone may be purchased from Shin-Etsu Silicones and has the trade name TMF 1.5, having the viscosity of 1.5 centistokes at 25° C.
2. Volatile Paraffinic Hydrocarbons

Also suitable as the volatile oils are various straight or branched chain paraffinic hydrocarbons having 5, 6, 7, 8, 9, 10, 11, 12, 13, 14, 15, 16, 17, 18, 19, or 20 carbon atoms, more preferably 8 to 16 carbon atoms. Suitable hydrocarbons include pentane, hexane, heptane, decane, dodecane, tetradecane, tridecane, and C₈₋₂₀ isoparaffins as disclosed in U.S. Pat. Nos. 3,439,088 and 3,818,105, both of which are hereby incorporated by reference in their entireties for all purposes.

Preferred volatile paraffinic hydrocarbons have a molecular weight of 70-225, preferably 160 to 190 and a boiling point range of 30 to 320, preferably 60 to 260° C., and a viscosity of less than about 10 cst. at 25° C. Such paraffinic hydrocarbons are available from EXXON under the ISOPARS trademark, and from the Permethyl Corporation. Suitable C₁₂ isoparaffins are manufactured by Permethyl Corporation under the tradename Permethyl 99A. Various C₁₆ isoparaffins commercially available, such as isohexadecane (having the tradename Permethyl R), are also suitable.

B. Non-Volatile Oils

A variety of nonvolatile oils are also suitable for use in the cosmetic compositions of the invention. The nonvolatile oils generally have a viscosity of greater than about 5 to 10 centistokes at 25° C., and may range in viscosity up to about 1,000,000 centipoise at 25° C. Preferably, the nonvolatile oils are liquid. Further eexamples of nonvolatile oils include, but are not limited to:
1. Esters

Suitable esters are mono-, di-, and triesters. The composition may comprise one or more esters selected from the group, or mixtures thereof.
(a) Monoesters

Monoesters are defined as esters formed by the reaction of a monocarboxylic acid having the formula R-COOH, wherein R is a straight or branched chain saturated or unsaturated alkyl having 2 to 45 carbon atoms, or phenyl; and an alcohol having the formula R-OH wherein R is a straight or branched chain saturated or unsaturated alkyl having 2-30 carbon atoms, or phenyl. Both the alcohol and the acid may be substituted with one or more hydroxyl groups. Either one or both of the acid or alcohol may be a “fatty” acid or alcohol, and may have from about 6 to 30 carbon atoms, more preferably 12, 14, 16, 18, or 22 carbon atoms in straight or branched chain, saturated or unsaturated form. Examples of monoester oils that may be used in the compositions of the invention include hexyl laurate, butyl isostearate, hexadecyl isostearate, cetyl palmitate, isostearyl neopentanoate, stearyl heptanoate, isostearyl isononanoate, steary lactate, stearyl octanoate, stearyl stearate, isononyl isononanoate, and so on.
(b) Diesters

Suitable diesters are the reaction product of a dicarboxylic acid and an aliphatic or aromatic alcohol or an aliphatic or aromatic alcohol having at least two substituted hydroxyl groups and a monocarboxylic acid. The dicarboxylic acid may contain from 2 to 30 carbon atoms, and may be in the straight or branched chain, saturated or unsaturated form. The dicarboxylic acid may be substituted with one or more hydroxyl groups. The aliphatic or aromatic alcohol may also contain 2 to 30 carbon atoms, and may be in the straight or branched chain, saturated, or unsaturated form. Preferably, one or more of the acid or alcohol is a fatty acid or alcohol, i.e. contains 12-22 carbon atoms. The dicarboxylic acid may also be an alpha hydroxy acid. The ester may be in the dimer or trimer form. Examples of diester oils that may be used in the compositions of the invention include diisotearyl malate, neopentyl glycol dioctanoate, dibutyl sebacate, dicetearyl dimer dilinoleate, dicetyl adipate, diisocetyl adipate, diisononyl adipate, diisostearyl dimer dilinoleate, diisostearyl fumarate, diisostearyl malate, dioctyl malate, and so on.
(c) Triesters

Suitable triesters comprise the reaction product of a tricarboxylic acid and an aliphatic or aromatic alcohol or alternatively the reaction product of an aliphatic or aromatic alcohol having three or more substituted hydroxyl groups with a monocarboxylic acid. As with the mono- and diesters mentioned above, the acid and alcohol contain 2 to 30 carbon atoms, and may be saturated or unsaturated, straight or branched chain, and may be substituted with one or more hydroxyl groups. Preferably, one or more of the acid or alcohol is a fatty acid or alcohol containing 12 to 22 carbon atoms. Examples of triesters include esters of arachidonic, citric, or behenic acids, such as triarachidin, tributyl citrate, triisostearyl citrate, tri C₁₂₋₁₃ alkyl citrate, tricaprylin, tricaprylyl citrate, tridecyl behenate, trioctyldodecyl citrate, tridecyl behenate; or tridecyl cocoate, tridecyl isononanoate, and so on.

Esters suitable for use in the composition are further described on pages 1670-1676 of the C.T.F.A. Cosmetic Ingredient Dictionary and Handbook, Eighth Edition, 2000, which is hereby incorporated by reference in its entirety.
2. Hydrocarbon Oils

It may be desirable to incorporate one or more nonvolatile hydrocarbon oils into the composition. Suitable nonvolatile hydrocarbon oils include paraffinic hydrocarbons and olefins, preferably those having greater than about 20 carbon atoms. Examples of such hydrocarbon oils include C₂₄₋₂₈ olefins, C₃₀₋₄₅ olefins, C₂₀₋₄₀ isoparaffins, hydrogenated polyisobutene, polyisobutene, polydecene, hydrogenated polydecene, mineral oil, pentahydrosqualene, squalene, squalane, and mixtures thereof. In one preferred embodiment such hydrocarbons have a molecular weight ranging from about 300 to 1000 Daltons.
3. Glyceryl Esters of Fatty Acids

Synthetic or naturally occurring glyceryl esters of fatty acids, or triglycerides, are also suitable for use in the compositions. Both vegetable and animal sources may be used. Examples of such oils include castor oil, lanolin oil, C₁₀₋₁₈ triglycerides, caprylic/capric/triglycerides, sweet almond oil, apricot kernel oil, sesame oil, camelina sativa oil, tamanu seed oil, coconut oil, corn oil, cottonseed oil, linseed oil, ink oil, olive oil, palm oil, illipe butter, rapeseed oil, soybean oil, grapeseed oil, sunflower seed oil, walnut oil, and the like.

Also suitable are synthetic or semi-synthetic glyceryl esters, such as fatty acid mono-, di-, and triglycerides which are natural fats or oils that have been modified, for example, mono-, di- or triesters of polyols such as glycerin. In an example, a fatty (C₁₂₋₂₂) carboxylic acid is reacted with one or more repeating glyceryl groups glyceryl stearate, diglyceryl diiosostearate, polyglyceryl-3 isostearate, polyglyceryl-4 isostearate, polyglyceryl-6 ricinoleate, glyceryl dioleate, glyceryl diisotearate, glyceryl tetraisostearate, glyceryl trioctanoate, diglyceryl distearate, glyceryl linoleate, glyceryl myristate, glyceryl isostearate, PEG castor oils, PEG glyceryl oleates, PEG glyceryl stearates, PEG glyceryl tallowates, and so on.
4. Nonvolatile Silicones

Nonvolatile silicone oils, both water soluble and water insoluble, are also suitable for use in the composition. Such silicones preferably have a viscosity ranging from about 10 to 800,000 cst, preferably 20 to 200,000 cst at 25° C. Suitable water insoluble silicones include amine functional silicones such as amodimethicone; phenyl substituted silicones such as bisphenylhexamethicone, trimethylsiloxyphenyl dimethicone, phenyl trimethicone, or polyphenylmethylsiloxane; dimethicone, dimethicone substituted with C₂₋₃₀ alkyl groups such cetyl dimethicone. Nonvolatile silicones may have the following general formula:

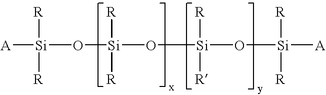

wherein R and R′ are each independently C₁₋₃₀ straight or branched chain, saturated or unsaturated alkyl, phenyl or aryl, trialkylsiloxy, and x and y are each independently 0-1,000,000; with the proviso that there is at least one of either x or y, and A is alkyl siloxy endcap unit. Preferred is where A is a methyl siloxy endcap unit; in particular trimethylsiloxy, and R and R′ are each independently a C₁₋₃₀ straight or branched chain alkyl, phenyl, or trimethylsiloxy, more preferably a C₁₋₂₂ alkyl, phenyl, or trimethylsiloxy, most preferably methyl, phenyl, or trimethylsiloxy, and resulting silicone is dimethicone, phenyl dimethicone, diphenyl dimethicone, phenyl trimethicone, or trimethylsiloxyphenyl dimethicone. Other examples include alkyl dimethicones such as cetyl dimethicone, and the like wherein at least one R is a fatty alkyl (C₁₂, C₁₄, C₁₆, C₁₈, C₂₀, or C₂₂), and the other R is methyl, and A is a trimethylsiloxy endcap unit, provided such alkyl dimethicone is a pourable liquid at room temperature. Phenyl trimethicone can be purchased from Dow Corning Corporation under the tradename 556 Fluid. Trimethylsiloxyphenyl dimethicone can be purchased from Wacker-Chemie under the tradename PDM-1000. Cetyl dimethicone, also referred to as a liquid silicone wax, may be purchased from Dow Corning as Fluid 2502, or from DeGussa Care & Surface Specialties under the tradenames Abil Wax 9801, or 9814.

C. Surfactants

The composition of the invention may contain one or more surfactants. The surfactants may be silicone or organic surfactants.
1. Silicone Surfactants

Suitable silicone surfactants include polyorganosiloxane polymers that have amphiphilic properties, for example contain both hydrophilic radicals and lipophilic radicals. These silicone surfactants may be liquids or solids at room temperature and include, but are not limited to those set forth herein.
(a) Dimethicone Copolyols or Alkyl Dimethicone Copolyols

One type of silicone surfactant that may be used is generally referred to as dimethicone copolyol or alkyl dimethicone copolyol. This surfactant is either a water-in-oil or oil-in-water surfactant having an Hydrophile/Lipophile Balance (HLB) ranging from about 2 to 18. Preferably the silicone surfactant is a nonionic surfactant having an HLB ranging from about 2 to 12, preferably about 2 to 10, most preferably about 4 to 6. The term “hydrophilic radical” means a radical that, when substituted onto the organosiloxane polymer backbone, confers hydrophilic properties to the substituted portion of the polymer. Examples of radicals that will confer hydrophilicity are hydroxy-polyethyleneoxy, hydroxyl, carboxylates, and mixtures thereof. The term “lipophilic radical” means an organic radical that, when substituted onto the organosiloxane polymer backbone, confers lipophilic properties to the substituted portion of the polymer. Examples of organic radicals that will confer lipophilicity are C₁₋₄₀ straight or branched chain alkyl, fluoro, aryl, aryloxy, C₁₋₄₀ hydrocarbyl acyl, hydroxy-polypropyleneoxy, or mixtures thereof. One type of suitable silicone surfactant has the general formula:

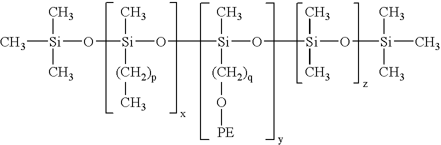

wherein p and q are from 0 to 40 (the range including all numbers between and subranges such as 2, 3, 4, 13, 14, 15, 16, 17, 18, etc.), and PE is (—C₂H₄O)ₐ—(—C₃H₆O)_{b}—H, wherein a is from 0 to 25, b is from 0 to 25, with the proviso that a and b cannot both be 0 simultaneously, wherein x, y and z are each independently ranging from 0 to 1 million, with the proviso that they cannot all be 0 simultaneously. In one preferred embodiment, x, y, z, a, and b are such that the molecular weight of the polymer ranges from about 5,000 to about 500,000, more preferably from about 10,000 to 100,000, and is most preferably approximately about 50,000 and the polymer is generically referred to as dimethicone copolyol.

One type of silicone surfactant is wherein p is such that the long chain alkyl is cetyl or lauryl, and the surfactant is called, generically, cetyl dimethicone copolyol or lauryl dimethicone copolyol respectively.

In some cases the number of repeating ethylene oxide or propylene oxide units in the polymer are also specified, such as a dimethicone copolyol that is also referred to as PEG-15/PPG-10 dimethicone, which refers to a dimethicone having substituents containing 15 ethylene glycol units and 10 propylene glycol units on the siloxane backbone. It is also possible for one or more of the methyl groups in the above general structure to be substituted with a longer chain alkyl (e.g. ethyl, propyl, butyl, etc.) or an ether such as methyl ether, ethyl ether, propyl ether, butyl ether, and the like.

Examples of silicone surfactants are those sold by Dow Corning under the tradename Dow Corning 3225C Formulation Aid having the CTFA name cyclotetrasiloxane (and) cyclopentasiloxane (and) PEG/PPG-18 dimethicone; or 5225C Formulation Aid, having the CTFA name cyclopentasiloxane (and) PEG/PPG-18/18 dimethicone; or Dow Coming 190 Surfactant having the CTFA name PEG/PPG-18/18 dimethicone; or Dow Corning 193 Fluid, Dow Corning 5200 having the CTFA name lauryl PEG/PPG-18/18 methicone; or Abil EM 90 having the CTFA name cetyl PEG/PPG-14/14 dimethicone sold by Goldschmidt; or Abil EM 97 having the CTFA name bis-cetyl PEG/PPG-14/14 dimethicone sold by Goldschmidt; or Abil WE 09 having the CTFA name cetyl PEG/PPG-10/1 dimethicone in a mixture also containing polyglyceryl-4 isostearate and hexyl laurate; or KF-6011 sold by Shin-Etsu Silicones having the CTFA name PEG-11 methyl ether dimethicone; KF-6012 sold by Shin-Etsu Silicones having the CTFA name PEG/PPG-20/22 butyl ether dimethicone; or KF-6013 sold by Shin-Etsu Silicones having the CTFA name PEG-9 dimethicone; or KF-6015 sold by Shin-Etsu Silicones having the CTFA name PEG-3 dimethicone; or KF-6016 sold by Shin-Etsu Silicones having the CTFA name PEG-9 methyl ether dimethicone; or KF-6017 sold by Shin-Etsu Silicones having the CTFA name PEG-10 dimethicone; or KF-6038 sold by Shin-Etsu Silicones having the CTFA name lauryl PEG-9 polydimethylsiloxyethyl dimethicone.
(b) Crosslinked Silicone Surfactants

Also suitable are various types of crosslinked silicone surfactants are referred to as emulsifying elastomers. They are typically prepared as set forth above with respect to the section “silicone elastomers” except that the silicone elastomers will contain at least one hydrophilic moiety such as polyoxyalkylenated groups. Typically these polyoxyalkylenated silicone elastomers are crosslinked organopolysiloxanes that may be obtained by a crosslinking addition reaction of diorganopolysiloxane comprising at least one hydrogen bonded to silicon and of a polyoxyalkylene comprising at least two ethylenically unsaturated groups. In at least one embodiment, the polyoxyalkylenated crosslinked organo-polysiloxanes are obtained by a crosslinking addition reaction of a diorganopolysiloxane comprising at least two hydrogens each bonded to a silicon, and a polyoxyalkylene comprising at least two ethylenically unsaturated groups, optionally in the presence of a platinum catalyst, as described, for example, in U.S. Pat. No. 5,236,986 and U.S. Pat. No. 5,412,004, U.S. Pat. No. 5,837,793 and U.S. Pat. No. 5,811,487, the contents of which are incorporated by reference.

Polyoxyalkylenated silicone elastomers that may be used in at least one embodiment of the invention include those sold by Shin-Etsu Silicones under the names KSG-21, KSG-20, KSG-30, KSG-31, KSG-32, KSG-33; KSG-210 which is dimethicone/PEG-10/15 crosspolymer dispersed in dimethicone; KSG-3 10 which is PEG-15 lauryl dimethicone crosspolymer; KSG-320 which is PEG-15 lauryl dimethicone crosspolymer dispersed in isododecane; KSG-330 (the former dispersed in triethylhexanoin), KSG-340 which is a mixture of PEG-10 lauryl dimethicone crosspolymer and PEG-15 lauryl dimethicone crosspolymer.

Also suitable are polyglycerolated silicone elastomers like those disclosed in PCT/WO 2004/024798, which is hereby incorporated by reference in its entirety. Such elastomers include Shin-Etsu's KSG series, such as KSG-710 which is dimethicone/polyglycerin-3 crosspolymer dispersed in dimethicone; or lauryl dimethicone/polyglycerin-3 crosspolymer dispersed in a variety of solvent such as isododecane, dimethicone, triethylhexanoin, sold under the Shin-Etsu tradenames KSG-810, KSG-820, KSG-830, or KSG-840. Also suitable are silicones sold by Dow Corning under the tradenames 9010 and DC9011.

One preferred crosslinked silicone elastomer emulsifier is dimethicone/PEG-10/15 crosspolymer.
2. Organic Surfactants

The composition may contain one or more additional surfactants, such as nonionic organic surfactants. Suitable nonionic surfactants include alkoxylated alcohols, or ethers, formed by the reaction of an alcohol with an alkylene oxide, usually ethylene or propylene oxide. Preferably the alcohol is either a fatty alcohol having 6 to 30 carbon atoms. Examples of such ingredients include Steareth 2-100, which is formed by the reaction of stearyl alcohol and ethylene oxide and the number of ethylene oxide units ranges from 2 to 100; Beheneth 5-30 which is formed by the reaction of behenyl alcohol and ethylene oxide where the number of repeating ethylene oxide units is 5 to 30; Ceteareth 2-100, formed by the reaction of a mixture of cetyl and stearyl alcohol with ethylene oxide, where the number of repeating ethylene oxide units in the molecule is 2 to 100; Ceteth 1-45 which is formed by the reaction of cetyl alcohol and ethylene oxide, and the number of repeating ethylene oxide units is 1 to 45, and so on.

Other alkoxylated alcohols are formed by the reaction of fatty acids and mono-, di- or polyhydric alcohols with an alkylene oxide. For example, the reaction products of C₆₋₃₀ fatty carboxylic acids and polyhydric alcohols which are monosaccharides such as glucose, galactose, methyl glucose, and the like, with an alkoxylated alcohol. Examples include polymeric alkylene glycols reacted with glyceryl fatty acid esters such as PEG glyceryl oleates, PEG glyceryl stearate; or PEG polyhydroxyalkanotes such as PEG dipolyhydroxystearate wherein the number of repeating ethylene glycol units ranges from 3 to 1000. Also suitable as nonionic surfactants are formed by the reaction of a carboxylic acid with an alkylene oxide or with a polymeric ether. The resulting products have the general formula: where RCO is the carboxylic ester radical, X is hydrogen or lower alkyl, and n is the number of polymerized alkoxy groups. In the case of the diesters, the two RCO-groups do not need to be identical. Preferably, R is a C₆₋₃₀ straight or branched chain, saturated or unsaturated alkyl, and n is from 1-100.

Monomeric, homopolymeric, or block copolymeric ethers are also suitable as nonionic surfactants. Typically, such ethers are formed by the polymerization of monomeric alkylene oxides, generally ethylene or propylene oxide. Such polymeric ethers have the following general formula: wherein R is H or lower alkyl and n is the number of repeating monomer units, and ranges from 1 to 500.

Other suitable nonionic surfactants include alkoxylated sorbitan and alkoxylated sorbitan derivatives. For example, alkoxylation, in particular ethoxylation of sorbitan provides polyalkoxylated sorbitan derivatives. Esterification of polyalkoxylated sorbitan provides sorbitan esters such as the polysorbates. For example, the polyalkyoxylated sorbitan can be esterified with C₆₋₃₀, preferably C₁₂₋₂₂ fatty acids. Examples of such ingredients include Polysorbates 20-85, sorbitan oleate, sorbitan sesquioleate, sorbitan palmitate, sorbitan sesquiisostearate, sorbitan stearate, and so on.

Certain types of amphoteric, zwitterionic, or cationic surfactants may also be used in the compositions. Descriptions of such surfactants are set forth in U.S. Pat. No. 5,843,193, which is hereby incorporated by reference in its entirety.

D. Oil Phase Structuring Agents

If desired, the composition may contain one or more oil phase structuring agents in the oil phase of the emulsion or anhydrous composition. The term “oil phase structuring agent” means an ingredient or combination of ingredients, soluble or dispersible in the oil phase, which will increase the viscosity, or structure, the oil phase. The structuring agent may be present in an amount sufficient to provide a liquid composition with increased viscosity, a semi-solid, or in some cases a solid composition that may be self-supporting. The structuring agent itself may be present in the liquid, semi-solid, or solid form. Suggested ranges of structuring agent are from about 0.01 to 70%, preferably from about 0.05 to 50%, more preferably from about 0.1-35% by weight of the total composition. Suitable oil phase structuring agents include those that are silicone based or organic based. They may be polymers or non-polymers, synthetic, natural, or a combination of both.
1. Silicone Structuring Agents

A variety of oil phase structuring agents may be silicone based, such as silicone elastomers, silicone gums, silicone waxes, linear silicones having a degree of polymerization that provides the silicone with a degree of viscosity such that when incorporated into the cosmetic composition it is capable of increasing the viscosity of the oil phase. Examples of silicone structuring agents include, but are not limited to:
(a) Silicone Elastomers

Silicone elastomers suitable for use in the compositions of the invention include those that are formed by addition reaction-curing, by reacting an SiH-containing diorganosiloxane and an organopolysiloxane having terminal olefinic unsaturation, or an alpha-omega diene hydrocarbon, in the presence of a platinum metal catalyst. Such elastomers may also be formed by other reaction methods such as condensation-curing organopolysiloxane compositions in the presence of an organotin compound via a dehydrogenation reaction between hydroxyl-terminated diorganopolysiloxane and SiH-containing diorganopolysiloxane or alpha omega diene; or by condensation-curing organopolysiloxane compositions in the presence of an organotin compound or a titanate ester using a condensation reaction between an hydroxyl-terminated diorganopolysiloxane and a hydrolysable organosiloxane; peroxide-curing organopolysiloxane compositions which thermally cure in the presence of an organoperoxide catalyst.

One type of elastomer that may be suitable is prepared by addition reaction-curing an organopolysiloxane having at least 2 lower alkenyl groups in each molecule or an alpha-omega diene; and an organopolysiloxane having at least 2 silicon-bonded hydrogen atoms in each molecule; and a platinum-type catalyst. While the lower alkenyl groups such as vinyl, can be present at any position in the molecule, terminal olefinic unsaturation on one or both molecular terminals is preferred. The molecular structure of this component may be straight chain, branched straight chain, cyclic, or network. These organopolysiloxanes are exemplified by methylvinylsiloxanes, methylvinylsiloxane-dimethylsiloxane copolymers, dimethylvinylsiloxy-terminated dimethylpolysiloxanes, dimethylvinylsiloxy-terminated dimethylsiloxane-methylphenylsiloxane copolymers, dimethylvinylsiloxy-terminated dimethylsiloxane-diphenylsiloxane-methylvinylsiloxane copolymers, trimethylsiloxy-terminated dimethylsiloxane-methylvinylsiloxane copolymers, trimethylsiloxy-terminated dimethylsiloxane-methylphenylsiloxane-methylvinylsiloxane copolymers, dimethylvinylsiloxy-terminated methyl(3,3,3-trifluoropropyl) polysiloxanes, and dimethylvinylsiloxy-terminated dimethylsiloxane-methyl(3,3,-trifluoropropyl)siloxane copolymers, decadiene, octadiene, heptadiene, hexadiene, pentadiene, or tetradiene, or tridiene.

Curing proceeds by the addition reaction of the silicon-bonded hydrogen atoms in the dimethyl methylhydrogen siloxane, with the siloxane or alpha-omega diene under catalysis using the catalyst mentioned herein. To form a highly crosslinked structure, the methyl hydrogen siloxane must contain at least 2 silicon-bonded hydrogen atoms in each molecule in order to optimize function as a crosslinker.

The catalyst used in the addition reaction of silicon-bonded hydrogen atoms and alkenyl groups, and is concretely exemplified by chloroplatinic acid, possibly dissolved in an alcohol or ketone and this solution optionally aged, chloroplatinic acid-olefin complexes, chloroplatinic acid-alkenylsiloxane complexes, chloroplatinic acid-diketone complexes, platinum black, and carrier-supported platinum.

Examples of suitable silicone elastomers for use in the compositions of the invention may be in the powder form, or dispersed or solubilized in solvents such as volatile or non-volatile silicones, or silicone compatible vehicles such as paraffinic hydrocarbons or esters. Examples of silicone elastomer powders include vinyl dimethicone/methicone silesquioxane crosspolymers like Shin-Etsu's KSP-100, KSP-101, KSP-102, KSP-103, KSP-104, KSP-105, hybrid silicone powders that contain a fluoroalkyl group like Shin-Etsu's KSP-200 which is a fluoro-silicone elastomer, and hybrid silicone powders that contain a phenyl group such as Shin-Etsu's KSP-300, which is a phenyl substituted silicone elastomer; and Dow Coming's DC 9506. Examples of silicone elastomer powders dispersed in a silicone compatible vehicle include dimethicone/vinyl dimethicone crosspolymers supplied by a variety of suppliers including Dow Corning Corporation under the tradenames 9040 or 9041, GE Silicones under the tradename SFE 839, or Shin-Etsu Silicones under the tradenames KSG-15, 16, 18. KSG-15 has the CTFA name cyclopentasiloxane/dimethicone/vinyl dimethicone crosspolymer. KSG-18 has the INCI name phenyl trimethicone/dimethicone/phenyl vinyl dimethicone crossoplymer. Silicone elastomers may also be purchased from Grant Industries under the Gransil trademark. Also suitable are silicone elastomers having long chain alkyl substitutions such as lauryl dimethicone/vinyl dimethicone crosspolymers supplied by Shin Etsu under the tradenames KSG-31, KSG-32, KSG-41, KSG-42, KSG-43, and KSG-44. Cross-linked organopolysiloxane elastomers useful in the present invention and processes for making them are further described in U.S. Pat. No. 4,970,252 to Sakuta et al., issued Nov. 13, 1990; U.S. Pat. No. 5,760,116 to Kilgour et al., issued Jun. 2, 1998; U.S. Pat. No. 5,654,362 to Schulz, Jr. et al. issued Aug. 5, 1997; and Japanese Patent Application JP 61-18708, assigned to Pola Kasei Kogyo KK, each of which are herein incorporated by reference in its entirety.
(b) Silicone Gums

Also suitable for use as an oil phase structuring agent are one or more silicone gums. The term “gum” means a silicone polymer having a degree of polymerization sufficient to provide a silicone having a gum-like texture. In certain cases the silicone polymer forming the gum may be crosslinked. The silicone gum typically has a viscosity ranging from about 500,000 to 100 million cst at 25° C., preferably from about 600,000 to 20 million, more preferably from about 600,000 to 12 million cst. All ranges mentioned herein include all subranges, e.g. 550,000; 925,000; 3.5 million.

The silicone gums that are used in the compositions include, but are not limited to, those of the general formula:

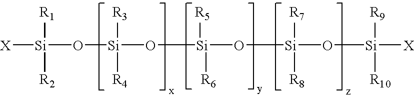

wherein, R₁ to R₉ are each independently an alkyl having 1 to 30 carbon atoms, aryl, or aralkyl; and X is OH or a C₁₋₃₀ alkyl, or vinyl; and wherein x, y, or z may be zero with the proviso that no more than two of x, y, or z are zero at any one time, and further that x, y, and z are such that the silicone gum has a viscosity of at least about 500,000 cst, ranging up to about 100 million centistokes at 25° C. Preferred is where R is methyl or OH.

Such silicone gums may be purchased in pure form from a variety of silicone manufacturers including Wacker-Chemie or Dow Corning, and the like. Such silicone gums include those sold by Wacker-Belsil under the trade names CM3092, Wacker-Belsil 1000, or Wacker-Belsil DM 3096. A silicone gum where X is OH, also referred to as dimethiconol, is available from Dow Coming Corporation under the trade name 1401. The silicone gum may also be purchased in the form of a solution or dispersion in a silicone compatible vehicle such as volatile or nonvolatile silicone. An example of such a mixture may be purchased from Barnet Silicones under the HL-88 tradename, having the INCI name dimethicone.
(c) Silicone Waxes

Another type of oily phase structuring agent includes silicone waxes that are typically referred to as alkyl silicone waxes which are semi-solids or solids at room temperature. The term “alkyl silicone wax” means a polydimethylsiloxane having a substituted long chain alkyl (such as C₁₆₋₃₀) that confers a semi-solid or solid property to the siloxane. Examples of such silicone waxes include stearyl dimethicone, which may be purchased from DeGussa Care & Surface Specialties under the tradename Abil Wax 9800 or from Dow Corning under the tradename 2503. Another example is bis-stearyl dimethicone, which may be purchased from Gransil Industries under the tradename Gransil A-18, or behenyl dimethicone, behenoxy dimethicone.
2. Polyamides or Silicone Polyamides

Also suitable as oil phase structuring agents are various types of polymeric compounds such as polyamides or silicone polyamides.

The term silicone polyamide means a polymer comprised of silicone monomers and monomers containing amide groups as further described herein. The silicone polyamide preferably comprises moieties of the general formula:

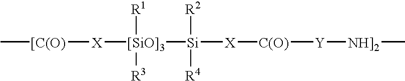

wherein, X is a linear or branched alkylene having from about 1-30 carbon atoms; R¹, R², R³, and R⁴ are each independently C₁₋₃₀ straight or branched chain alkyl which may be substituted with one or more hydroxyl or halogen groups; phenyl which may be substituted with one or more C₁₋₃₀ alkyl groups, halogen, hydroxyl, or alkoxy groups; or a siloxane chain having the general formula:

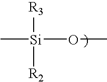

and Y is either
(a) a linear or branched alkylene having from about 1-40 carbon atoms which may be substituted with (i) one or more amide groups having the general formula R₁CONRI, or (ii) C₅₋₆ cyclic ring, or (iii) phenylene which may be substituted with one or more C₁₋₁₀ alkyl groups, or (iv) hydroxy, or (v) C₃₋₈ cycloalkane, or (vi) C₁₋₂₀ alkyl which may be substituted with one or more hydroxy groups, or (vii) C₁₋₁₀ alkyl amines; or
(b) TR₅R₆R₇ wherein R₅, R₆, and R₇, are each independently a C₁₋₁₀ linear or branched alkylenes and T is CR₈ wherein R₈ is hydrogen, a trivalent atom N, P, or Al, or a C₁₋₃₀ straight or branched chain alkyl which may be substituted with one or more hydroxyl or halogen groups; phenyl which may be substituted with one or more C₁₋₃₀ alkyl groups, halogen, hydroxyl, or alkoxy groups; or a siloxane chain having the general formula:

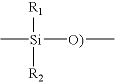

Preferred is where R₁, R₂, R₃, and R₄ are C₁₋₁₀, preferably methyl; and X and Y is a linear or branched alkylene. Preferred are silicone polyamides having the general formula

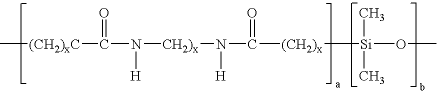

wherein a and b are each independently sufficient to provide a silicone polyamide polymer having a melting point ranging from about 60 to 120° C., and a molecular weight ranging from about 40,000 to 500,000 Daltons. One type of silicone polyamide that may be used in the compositions of the invention may be purchased from Dow Corning Corporation under the tradename Dow Coming 2-8178 gellant which has the CTFA name nylon-611/dimethicone copolymer which is sold in a composition containing PPG-3 myristyl ether.

Also suitable are polyamides such as those purchased from Arizona Chemical under the tradenames Uniclear and Sylvaclear. Such polyamides may be ester terminated or amide terminated. Examples of ester terminated polyamides include, but are not limited to those having the general formula:

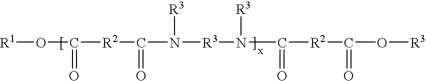

wherein n denotes a number of amide units such that the number of ester groups ranges from about 10% to 50% of the total number of ester and amide groups; each R¹ is independently an alkyl or alkenyl group containing at least 4 carbon atoms; each R² is independently a C₄₋₄₂ hydrocarbon group, with the proviso that at least 50% of the R² groups are a C₃₀₋₄₂ hydrocarbon; each R³ is independently an organic group containing at least 2 carbon atoms, hydrogen atoms and optionally one or more oxygen or nitrogen atoms; and each R⁴ is independently a hydrogen atom, a C₁₋₁₀ alkyl group or a direct bond to R³ or to another R⁴, such that the nitrogen atom to which R³ and R⁴ are both attached forms part of a heterocyclic structure defined by R⁴-N-R³, with at least 50% of the groups R⁴ representing a hydrogen atom.

General examples of ester and amide terminated polyamides that may be used as oil phase gelling agents include those sold by Arizona Chemical under the tradenames Sylvaclear A200V or A2614V, both having the CTFA name ethylenediamine/hydrogenated dimer dilinoleate copolymer/bis-di-C₁₄₋₁₈ alkyl amide; Sylvaclear AF1900V; Sylvaclear C75V having the CTFA name bis-stearyl ethylenediamine/neopentyl glycol/stearyl hydrogenated dimer dilinoleate copolymer; Sylvaclear PA1200V having the CTFA name Polyamide-3; Sylvaclear PE400V; Sylvaclear WF1500V; or Uniclear, such as Uniclear 100VG having the INCI name ethylenediamine/stearyl dimer dilinoleate copolymer; or ethylenediamine/stearyl dimer ditallate copolymer. Other examples of suitable polyamides include those sold by Henkel under the Versamid trademark (such as Versamid 930, 744, 1655), or by Olin Mathieson Chemical Corp. under the brand name Onamid S or Onamid C.
3. Natural or Synthetic Organic Waxes

Also suitable as the oil phase structuring agent may be one or more natural or synthetic waxes such as animal, vegetable, or mineral waxes. Preferably such waxes will have a higher melting point such as from about 60 to 150° C., more preferably from about 65 to 100° C. Examples of such waxes include waxes made by Fischer-Tropsch synthesis, such as polyethylene or synthetic wax; or various vegetable waxes such as bayberry, candelilla, ozokerite, acacia, beeswax, ceresin, cetyl esters, flower wax, citrus wax, carnauba wax, jojoba wax, japan wax, polyethylene, microcrystalline, rice bran, lanolin wax, mink, montan, bayberry, ouricury, ozokerite, palm kernel wax, paraffin, avocado wax, apple wax, shellac wax, clary wax, spent grain wax, grape wax, and polyalkylene glycol derivatives thereof such as PEG6-20 beeswax, or PEG-12 carnauba wax; or fatty acids or fatty alcohols, including esters thereof, such as hydroxystearic acids (for example 12-hydroxy stearic acid), tristearin, tribehenin, and so on.
4. Montmorillonite Minerals

One type of structuring agent that may be used in the composition comprises natural or synthetic montmorillonite minerals such as hectorite, bentonite, and quaternized derivatives thereof, which are obtained by reacting the minerals with a quaternary ammonium compound, such as stearalkonium bentonite, hectorites, quaternized hectorites such as Quaternium-18 hectorite, attapulgite, carbonates such as propylene carbonate, bentones, and the like.
5. Silicas and Silicates

Another type of structuring agent that may be used in the oil phase of the composition is silica, silicates, or silica silylate, and alkali metal or alkaline earth metal derivatives thereof. These silicas and silicates are generally found in the particulate form and include silica, silica silylate, magnesium aluminum silicate, and the like.

E. Humectants

It may also be desirable to include one or more humectants in the composition. If present, such humectants may range from about 0.001 to 25%, preferably from about 0.005 to 20%, more preferably from about 0.1 to 15% by weight of the total composition. Examples of suitable humectants include glycols in monomeric or polymeric form such as polyethylene and polypropylene glycols such as PEG 4-200, which are polyethylene glycols having from 4 to 200 repeating ethylene oxide units; as well as C₁₋₆ alkylene glycols such as propylene glycol, butylene glycol, pentylene glycol, and the like. Preferably, the humectants used in the composition of the invention are C₁₋₆, preferably C₂₋₄ alkylene glycols, most particularly butylene glycol.

F. Aqueous Phase Structuring Agents

If the compositions of the invention contain an aqueous phase it may be desirable to include one or more aqueous phase structuring agents in the composition. Such agents will typically thicken or increase the viscosity of the aqueous phase. If present, suggested ranges are from about 0.01 to 30%, preferably from about 0.1 to 20%, more preferably from about 0.5 to 15% by weight of the total composition. Examples of such agents include various acrylate based thickening agents, natural or synthetic gums, and the like.
1. Acrylate Polymers

For example, acrylic polymeric thickeners comprised of monomers A and B wherein A is selected from the group consisting of acrylic acid, methacrylic acid, and mixtures thereof, and B is selected from the group consisting of a C₁₋₂₂ alkyl acrylate, a C₁₋₂₂ alky methacrylate, and mixtures thereof are suitable. In one embodiment the A monomer comprises one or more of acrylic acid or methacrylic acid, and the B monomer is selected from the group consisting of a C₁₋₁₀, most preferably C₁₋₄ alkyl acrylate, a C₁₋₁₀, most preferably C₁₋₄ alkyl methacrylate, and mixtures thereof. Most preferably the B monomer is one or more of methyl or ethyl acrylate or methacrylate. The acrylic copolymer may be supplied in an aqueous solution having a solids content ranging from about 10-60%, preferably 20-50%, more preferably 25-45% by weight of the polymer, with the remainder water. The composition of the acrylic copolymer may contain from about 0.1-99 parts of the A monomer, and about 0.1-99 parts of the B monomer. Acrylic polymer solutions include those sold by Seppic, Inc., under the tradename Capigel.

Also suitable are acrylic polymeric thickeners that are copolymer of A, B, and C monomers wherein A and B are as defined above, and C has the general formula:

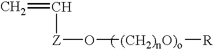

wherein Z is —(CH₂)ₘ; wherein m is 1-10, n is 2-3, o is 2-200, and R is a C₁₀₋₃₀ straight or branched chain alkyl. Examples of the secondary thickening agent above, are copolymers where A and B are defined as above, and C is CO, and wherein n, o, and R are as above defined. Examples of such secondary thickening agents include acrylates/steareth-20 methacrylate copolymer, which is sold by Rohm & Haas under the tradename Acrysol ICS-1.

Also suitable are acrylate based anionic amphiphilic polymers containing at least one hydrophilic unit and at least one allyl ether unit containing a fatty chain. Preferred are those where the hydrophilic unit contains an ethylenically unsaturated anionic monomer, more specificially a vinyl carboxylic acid such as acrylic acid, methacrylic acid or mixtures thereof, and where the allyl ether unit containing a fatty chain corresponds to the monomer of formula:

CH₂=CR′CH₂OBₙR

in which R′ denotes H or CH₃, B denotes the ethylenoxy radical, n is zero or an integer ranging from 1 to 100, R denotes a hydrocarbon radical selected from alkyl, arylalkyl, aryl, alkylaryl and cycloalkyl radicals which contain from 8 to 30 carbon atoms, preferably from 10 to 24, and even more particularly from 12 to 18 carbon atoms. More preferred in this case is where R′ denotes H, n is equal to 10 and R denotes a stearyl (C₁₈) radical. Anionic amphiphilic polymers of this type are described and prepared in U.S. Pat. Nos. 4,677,152 and 4,702,844, both of which are hereby incorporated by reference in their entirety. Among these anionic amphiphilic polymers, polymers formed of 20 to 60% by weight acrylic acid and/or methacrylic acid, of 5 to 60% by weight lower alkyl methacrylates, of 2 to 50% by weight allyl ether containing a fatty chain as mentioned above, and of 0 to 1% by weight of a crosslinking agent which is a well-known copolymerizable polyethylenic unsaturated monomer, for instance diallyl phthalate, allyl (meth)acrylate, divinylbenzene, (poly)ethylene glycol dimethacrylate and methylenebis-acrylamide. One commercial example of such polymers are crosslinked terpolymers of methacrylic acid, of ethyl acrylate, of polyethylene glycol (having 10 EO units) ether of stearyl alcohol or steareth-10, in particular those sold by the company Allied Colloids under the names SALCARE SC80 and SALCARE SC90, which are aqueous emulsions containing 30% of a crosslinked terpolymer of methacrylic acid, of ethyl acrylate and of steareth-10 allyl ether (40/50/10).

Also suitable are acrylate copolymers such as Polyacrylate-3 which is a copolymer of methacrylic acid, methylmethacrylate, methylstyrene isopropylisocyanate, and PEG-40 behenate monomers; Polyacrylate-10 which is a copolymer of sodium acryloyldimethyltaurate, sodium acrylate, acrylamide and vinyl pyrrolidone monomers; or Polyacrylate-11, which is a copolymer of sodium acryloyldimethylacryloyldimethyl taurate, sodium acrylate, hydroxyethyl acrylate, lauryl acrylate, butyl acrylate, and acrylamide monomers.

Also suitable are crosslinked acrylate based polymers where one or more of the acrylic groups may have substituted long chain alkyl groups (such as 6-40, 10-30, and the like), for example acrylates/C₁₀₋₃₀ alkyl acrylate crosspolymer which is a copolymer of C₁₀₋₃₀ alkyl acrylate and one or more monomers of acrylic acid, methacrylic acid, or one of their simple esters crosslinked with the allyl ether of sucrose or the allyl ether of pentaerythritol. Such polymers are commonly sold under the Carbopol or Pemulen tradenames.

Particularly suitable as the aqueous phase thickening agent are acrylate based polymeric thickeners sold by Clariant under the Aristoflex trademark such as Aristoflex AVC, which is ammonium acryloyldimethyltaurate/VP copolymer; Aristoflex AVL which is the same polymer has found in AVC dispersed in mixture containing caprylic/capric triglyceride, trilaureth-4, and polyglyceryl-2 sesquiisostearate; or Aristoflex HMB which is ammonium acryloyldimethyltaurate/beheneth-25 methacrylate crosspolymer, and the like.
2. High Molecular Weight PEG or Polyglycerins

Also suitable as the aqueous phase thickening agents are various polyethylene glycols (PEG) derivatives where the degree of polymerization ranges from 1,000 to 200,000. Such ingredients are indicated by the designation “PEG” followed by the degree of polymerization in thousands, such as PEG-45M, which means PEG having 45,000 repeating ethylene oxide units. Examples of suitable PEG derivatives include PEG 2M, 5M, 7M, 9M, 14M, 20M, 23M, 25M, 45M, 65M, 90M, 115M, 160M, 180M, and the like.

Also suitable are polyglycerins which are repeating glycerin moieties where the number of repeating moieties ranges from 15 to 200, preferably from about 20-100. Examples of suitable polyglycerins include those having the CFTA names polyglycerin-20, polyglycerin-40, and the like.
3. Polysaccharides

Also suitable as aqueous phase thickening agents are various types of polysaccharides, such as xanthan gum, cellulose, dextrin, cyclodextrin, hydroxyethylcellulose, acacia gum, and the like.

G. Additional Botanical Extracts

It may be desirable to include one or more botanical extracts in the compositions in addition to those botanical extracts that have kinase inhibitor activity. If so, suggested ranges are from about 0.0001 to 10%, preferably about 0.0005 to 8%, more preferably about 0.001 to 5% by weight of the total composition. Suitable botanical extracts include extracts from plants (herbs, roots, flowers, fruits, seeds) such as flowers, fruits, vegetables, and so on, including yeast ferment extract, padica pavonica extract, thermus thermophilis ferment extract, camelina sativa seed oil, boswellia serrata extract, olive extract, aribodopsis thaliana extract, acacia dealbata extract, acer saccharinum (sugar maple), acidopholus, acorus, aesculus, agaricus, agave, agrimonia, algae, aloe, citrus, brassica, cinnamon, orange, apple, blueberry, cranberry, peach, pear, lemon, lime, pea, seaweed, caffeine, green tea, chamomile, willowbark, mulberry, rosemary, poppy, and the like. Further specific examples include, but are not limited to, Glycyrrhiza Glabra, Salix Nigra, Macrocycstis Pyrifera, Pyrus Malus, Saxifraga Sarmentosa, Vilis Vinifera, Morus Nigra, Scutellaria Baicalensis, Anthemis Nobilis, Salvia Sclarea, Citrus Medica Limonum, Panax Ginseng, and mixtures thereof.

H. Sunscreens

In some cases it may be desirable to add sunscreens to the self tanning compositions. Such sunscreens include chemical UVA or UVB sunscreens or physical sunscreens in the particulate form.
1. UVA Chemical Sunscreens

If desired, the composition may comprise one or more UVA sunscreens. The term “UVA sunscreen” means a chemical compound that blocks UV radiation mainly in the wavelength range of about 320 to 400 nm. Preferred UVA sunscreens are dibenzoylmethane compounds having the general formula

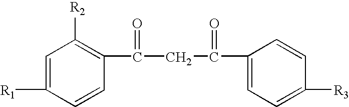

wherein R₁ is H, OR and NRR wherein each R is independently H, C₁₋₂₀ straight or branched chain alkyl; R₂ is H or OH; and R₃ is H, C₁₋₂₀ straight or branched chain alkyl.

Preferred is where R₁ is OR where R is a C₁₋₂₀ straight or branched alkyl, preferably methyl; R₂ is H; and R₃ is a C₁₋₂₀ straight or branched chain alkyl, more preferably, butyl. Examples of suitable UVA sunscreen compounds of this general formula include 4-methyldibenzoylmethane, 2-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4′diisopropylbenzoylmethane, 4-tert-butyl-4′-methoxydibenzoylmethane, 4,4′-diisopropylbenzoylmethane, 2-methyl-5-isopropyl-4′-methoxydibenzoymethane, 2-methyl-5-tert-butyl-4′-methoxydibenzoylmethane, and so on. Particularly preferred is 4-tert-butyl-4′-methoxydibenzoylmethane, also referred to as Avobenzone. Avobenzone is commercial available from Givaudan-Roure under the trademark Parsol 1789, and Merck & Co. under the tradename Eusolex 9020.

The composition may contain from about 0.001-20%, preferably 0.005-5%, more preferably about 0.005-3% by weight of the composition of UVA sunscreen. In the preferred embodiment of the invention the UVA sunscreen is Avobenzone, and it is present at not greater than about 3% by weight of the total composition.
2. UVB Chemical Sunscreens

The term “UVB sunscreen” means a compound that absorbs UV radiation mainly in the wavelength range of from about 290 to 320 nm. A variety of UVB chemical sunscreens exist including alpha-cyano-beta,beta-diphenyl acrylic acid esters as set forth in U.S. Pat. No. 3,215,724, which is hereby incorporated by reference in its entirety. One particular example of an alpha-cyano-beta,beta-diphenyl acrylic acid ester is Octocrylene, which is 2-ethylhexyl 2-cyano-3,3-diphenylacrylate. In certain cases the composition may contain no more than about 110% by weight of the total composition of octocrylene. Suitable amounts range from about 0.001-10% by weight. Octocrylene may be purchased from BASF under the tradename Uvinul N-539.

Other suitable sunscreens include benzylidene camphor derivatives as set forth in U.S. Pat. No. 3,781,417, which is hereby incorporated by reference in its entirety. Such benzylidene camphor derivatives have the general formula:

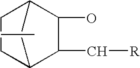

wherein R is p-tolyl or styryl, preferably styryl. Particularly preferred is 4-methylbenzylidene camphor, which is a lipid soluble UVB sunscreen compound sold under the tradename Eusolex 6300 by Merck.

Also suitable are cinnamate derivatives having the general formula:

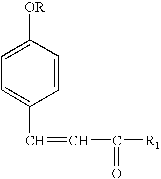

wherein R and R₁ are each independently a C₁₋₂₀ straight or branched chain alkyl. Preferred is where R is methyl and R₁ is a branched chain C₁₋₁₀, preferably C₈ alkyl. The preferred compound is ethylhexyl methoxycinnamate, also referred to as Octoxinate or octyl methoxycinnamate. The compound may be purchased from Givaudan Corporation under the tradename Parsol MCX, or BASF under the tradename Uvinul MC 80. Also suitable are mono-, di-, and triethanolamine derivatives of such methoxy cinnamates including diethanolamine methoxycinnamate. Cinoxate, the aromatic ether derivative of the above compound is also acceptable.

Also suitable as UVB screening agents are various benzophenone derivatives having the general formula:

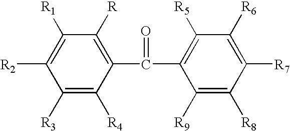

wherein R through R₉ are each independently H, OH, NaO₃S, SO₃H, SO₃Na, Cl, R″, OR″ where R″ is C₁₋₂₀ straight or branched chain alkyl. Examples of such compounds include Benzophenone 1, 2, 3, 4, 5, 6, 7, 8, 9, 10, 11, and 12. Particularly preferred is where the benzophenone derivative is Benzophenone 3 (also referred to as Oxybenzone), Benzophenone 4 (also referred to as Sulisobenzone), Benzophenone 5 (Sulisobenzone Sodium), and the like. Most preferred is Benzophenone 3.

Also suitable are certain menthyl salicylate derivatives having the general formula:

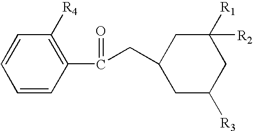

wherein R₁, R₂, R₃, and R₄ are each independently H, OH, NH₂, or C₁₋₂₀ straight or branched chain alkyl. Particularly preferred is where R₁, R₂, and R₃ are methyl and R₄ is hydroxyl or NH₂, the compound having the name homomenthyl salicylate (also known as Homosalate) or menthyl anthranilate. Homosalate is available commercially from Merck under the tradename Eusolex HMS and menthyl anthranilate is commercially available from Haarmann & Reimer under the tradename Heliopan. If present, the Homosalate should be found at no more than about 15% by weight of the total composition.

Various amino benzoic acid derivatives are suitable UVB absorbers including those having the general formula:

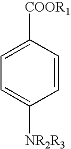

wherein R₁, R₂, and R₃ are each independently H, C₁₋₂₀ straight or branched chain alkyl which may be substituted with one or more hydroxy groups. Particularly preferred is wherein R₁ is H or C₁₋₈ straight or branched alkyl, and R₂ and R₃ are H, or C₁₋₈ straight or branched chain alkyl. Particularly preferred are PABA, ethyl hexyl dimethyl PABA (Padimate O), ethyldihydroxypropyl PABA, and the like. If present Padimate O should be found at no more than about 8% by weight of the total composition.

Salicylate derivatives are also acceptable UVB absorbers. Such compounds have the general formula: wherein R is a straight or branched chain alkyl, including derivatives of the above compound formed from mono-, di-, or triethanolamines. Particular preferred are octyl salicylate, TEA-salicylate, DEA-salicylate, and mixtures thereof.

Generally, the amount of the UVB chemical sunscreen present may range from about 0.001-45%, preferably 0.005-40%, more preferably about 0.01-35% by weight of the total composition.

If desired, the compositions of the invention may be formulated to have a certain SPF (sun protective factor) values ranging from about 1-50, preferably about 2-45, most preferably about 5-30. Calculation of SPF values is well known in the art. Preferably, the claimed compositions have SPF values greater than 4.

I. Particulate Materials

The compositions of the invention may contain particulate materials in the form of pigments, inert particulates, or mixtures thereof. If present, suggested ranges are from about 0.1-75%, preferably about 0.5-70%, more preferably about 0.1-65% by weight of the total composition. In the case where the composition may comprise mixtures of pigments and powders, suitable ranges include about 0.01-75% pigment and 0.1-75% powder, such weights by weight of the total composition.
1. Powders

The particulate matter may be colored or non-colored (for example white) non-pigmentatious powders. Suitable non-pigmentatious powders include bismuth oxychloride, titanated mica, fumed silica, spherical silica, polymethylmethacrylate, micronized teflon, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, microcrystalline cellulose, rice starch, silica, talc, mica, titanium dioxide, zinc laurate, zinc myristate, zinc rosinate, alumina, attapulgite, calcium carbonate, calcium silicate, dextran, kaolin, nylon, silica silylate, sericite, soy flour, tin oxide, titanium hydroxide, trimagnesium phosphate, walnut shell powder, or mixtures thereof. The above mentioned powders may be surface treated with lecithin, amino acids, mineral oil, silicone, or various other agents either alone or in combination, which coat the powder surface and render the particles more lipophilic in nature.
2. Pigments

The particulate materials may comprise various organic and/or inorganic pigments. The organic pigments are generally various aromatic types including azo, indigoid, triphenylmethane, anthroquinone, and xanthine dyes which are designated as D&C and FD&C blues, browns, greens, oranges, reds, yellows, etc. Organic pigments generally consist of insoluble metallic salts of certified color additives, referred to as the Lakes. Inorganic pigments include iron oxides, ultramarines, chromium, chromium hydroxide colors, and mixtures thereof. Iron oxides of red, blue, yellow, brown, black, and mixtures thereof are suitable.

J. Preservatives

The composition may contain 0.001-8%, preferably 0.01-6%, more preferably 0.05-5% by weight of the total composition of preservatives. A variety of preservatives are suitable, including such as benzoic acid, benzyl alcohol, benzylhemiformal, benzylparaben, 5-bromo-5-nitro-1,3-dioxane, 2-bromo-2-nitropropane-1,3-diol, butyl paraben, phenoxyethanol, methyl paraben, propyl paraben, diazolidinyl urea, calcium benzoate, calcium propionate, caprylyl glycol, biguanide derivatives, phenoxyethanol, captan, chlorhexidine diacetate, chlorhexidine digluconate, chlorhexidine dihydrochloride, chloroacetamide, chlorobutanol, p-chloro-m-cresol, chlorophene, chlorothymol, chloroxylenol, m-cresol, o-cresol, DEDM Hydantoin, DEDM Hydantoin dilaurate, dehydroacetic acid, dibromopropamidine diisethionate, DMDM Hydantoin, and the like. In one preferred embodiment the composition is free of parabens.

K. Vitamins and Antioxidants

The compositions of the invention, may contain vitamins and/or coenzymes, as well as antioxidants. If so, 0.001-10%, preferably 0.01-8%, more preferably 0.05-5% by weight of the total composition are suggested. Suitable vitamins include ascorbic acid and derivatives thereof, the B vitamins such as thiamine, riboflavin, pyridoxin, and so on, as well as coenzymes such as thiamine pyrophoshate, flavin adenin dinucleotide, folic acid, pyridoxal phosphate, tetrahydrofolic acid, and so on. Also Vitamin A and derivatives thereof are suitable. Examples are Vitamin A palmitate, acetate, or other esters thereof, as well as Vitamin A in the form of beta carotene. Also suitable is Vitamin E and derivatives thereof such as Vitamin E acetate, nicotinate, or other esters thereof. In addition, Vitamins D and K are suitable.

Suitable antioxidants are ingredients which assist in preventing or retarding spoilage. Examples of antioxidants suitable for use in the compositions of the invention are potassium sulfite, sodium bisulfite, sodium erythrobate, sodium metabisulfite, sodium sulfite, propyl gallate, cysteine hydrochloride, butylated hydroxytoluene, butylated hydroxyanisole, and so on.

The invention will be further described in connection with the following examples which are set forth for the purposes of illustration only.

### EXAMPLE 1

| | % by weight | | |
|---|---|---|---|
| | Self-tan | Self-tan | Self-tan |
| Ingredients | spray | Lotion (dark) | Lotion (medium) |
| Ethoxydiglycol | 10.00 | | |
| Dimethicone 20 centistokes | | 1.00 | 1.00 |
| Cyclopentasiloxane | | 12.03 | 12.03 |
| Cyclopentasiloxane/dimethicone/vinyl | | 4.00 | 4.00 |
| dimethicone crosspolymer | | | |
| Cyclopentasiloxane PEG/PPG- | | 5.00 | 5.00 |
| 18/18 dimethicone | | | |
| Stearyl dimethicone | | 0.50 | 0.50 |
| Dimethicone (Barnet, HL-88) | | 0.50 | 0.50 |
| Cetyl PEG/PPG-10/1 dimethicone | | 1.00 | 1.00 |
| PEG-12 laurate | 1.50 | | |
| Squalane/helianthus annuus | | 0.10 | 0.10 |
| (sunflower) seed oil/hibiscus | | | |
| abelmoschuus extract | | | |
| Methyl glucose dioleate | | 0.20 | 0.20 |
| Caprylic/capric/myristic/stearic triglyceride | | 0.50 | 0.50 |
| BHT | | 0.05 | 0.05 |
| Bisabolol | | 0.10 | 0.10 |
| Lactic acid | | | 1.20 |
| PEG-40 castor oil | 1.00 | | |
| Fragrance | 0.53 | 0.40 | 0.40 |
| Tocopheryl acetate | | 0.01 | 0.01 |
| Isopentyldiol | | 3.00 | 3.00 |
| Phenoxyethanol/methylparaben/ | | 0.50 | 0.50 |
| ethylparaben/Butyl paraben/ | | | |
| propylparaben/isobutyl paraben | | | |
| Dihydroxyacetone | | 5.00 | 7.00 |
| Lactic acid | | 1.20 | |
| Water | QS | QS | QS |
| Pantethine | | 0.01 | 0.01 |
| Butylene glycol | 3.00 | | |
| PPG-12-buteth-16 | 0.50 | | |
| Nylon-12 | | 0.50 | 0.50 |
| Citric acid | | 0.20 | 0.20 |
| Cyclodextrin | | 1.50 | 1.50 |
| Sodium chloride | | 0.80 | 0.80 |
| Sucrose | | | 2.00 |
| Fructose | | | 2.00 |
| Dimethyl isosorbide | 10.00 | | |
| Hydrolyzed wheat | 0.20 | | |
| protein/hydrolyzed wheat starch | | | |
| Methyldibromo | 0.20 | | |
| glutaronitrile/phenoxyethanol | | | |
| Dihydroxyacetone | 5.00 | | |
| Glycerth-7 triacetate | 5.00 | | |
| Glyceryl-3 phosphate | 1.00 | | |
| Betula Alba extract | | 1.00 | |
| Genestein | | | 0.50 |

The compositions were prepared by combining the oil and water phases separately, then mixing well to emulsify.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

We claim:

1. A self tanning cosmetic composition comprising at least one skin tanning agent and at least one kinase inhibitor operable to inhibit the kinase induced phosphorylation of the skin tanning agent when the composition is applied to skin.

2. The composition of claim 1 wherein skin tanning agent is DHA.

3. The composition of claim 1 wherein the skin tanning agent is DHA either alone or in combination with glucose, fructose, erythrulose, xylose, or mixtures thereof.

4. The composition of claim 1 wherein the skin tanning agent is present in an amount ranging from about 0.001-50%, and the kinase inhibitor is present in an amount ranging from about 0.0001 to 25%, all percentage by weight of the total composition, and the kinase inhibitor is a peptide, botanical extract, compound, or mixtures thereof.

5. The composition of claim 4 wherein the kinase inhibitor is a chemical compound which is glyceryl-3 phosphate.

6. A method for increasing the efficacy of DHA in self tanning compositions comprising formulating the composition with at least one kinase inhibitor operable to inhibit the kinase induced phosphorylation of DHA.

7. The method of claim 6 wherein the composition additionally comprises an ingredient selected from the group consisting of glucose, fructose, erythrulose, xylose, and mixtures thereof.

8. The method of claim 6 wherein the kinase inhibitor is added in an amount ranging from about 0.0001 to 25% by weight of the total composition.

9. The method of claim 6 wherein the self tanning composition is in the form of a liquid spray or semi-solid cream.

10. The method of claim 6 wherein the composition is in the form of an aqueous emulsion further comprising water, at least one oil, at least one structuring agent, and at least one surfactant.

11. A self tanning composition comprising at least one skin tanning agent operable to darken skin due to the Maillard Reaction, and at least one kinase inhibitor derived from a botanical extract having greater than about 0.1% ursolic acid content, which is operable to inhibit the kinase induced phosphorylation of DHA.

12. The composition of claim 11 wherein the botanical extract is selected from the group consisting of an:
extract obtained from the botanical Nonomuraea longicatena, also referred to as K252a,
extract from Cladosporium cladosporioides, also referred to as Calphostin C,
extract from Hypericum perforatum, also known as Hypericin,
extract from Ligustrum Lucidum extract,
extract from Eriobotrya Japonica extract,
Loquat Leaf Extract,
Betula Alba extract,
Fucus Vesiculosis (Bladderwrack) Extract,
Laminaria Digitata (Atlantic Kelp) Extract,
Origanum Majorana Extract,
Polylepsis Racemosa Extract,
Oldenlandia Diffusa Extract,
Leocerus Cardiaca (Motherwart) Extract,
Ocimum Tenuiflorum (Sacred Basil) Extract,
Pygeum Africanum Extract,
Frangula Bark Extract (6-Methyl-1,3,8-tri [symbol] hydroxy [symbol] anthra [symbol] quinine); and
mixtures thereof.

13. The composition of claim 11 which is in the emulsion form and contains at least one aqueous phase structuring agent which is a polysaccharide.

14. The composition of claim 11 which further comprises a silicone elastomer.

15. The composition of claim 11 which further comprises a nonvolatile silicone.

16. A method for darkening the color of skin to provide the appearance of a tan by applying to the skin a composition comprising at least one skin tanning agent reactive with phosphorus and at least one kinase inhibitor operable to inhibit the kinase induced phosphorylation of the self tanning agent when the composition is applied to the skin.

17. The method of claim 16 wherein the skin tanning composition is applied to the skin in the form of a spray, lotion, creme, gel, or mousse.

18. The method of claim 17 wherein the skin tanning agent is in the form of an emulsion.

19. The method of claim 16 wherein the skin tanning agent is DHA.

20. The method of claim 16 wherein the kinase inhibitor is a botanical extract.
